(19) United States
(12) Patent Application Publication
(10) Pub. No.: US 2022/0104874 A1
(43) Pub. Date: Apr. 7, 2022

(54) ELONGATED MEDICAL ASSEMBLY

(71) Applicant: Baylis Medical Company Inc., Montreal (CA)

(72) Inventors: Jackie Leung, Richmond Hill (CA); Eduardo Moriyama, Richmond (CA); Gareth Davies, Toronto (CA); Kaylie Lau, Toronto (CA)

(21) Appl. No.: 17/465,047

(22) Filed: Sep. 2, 2021

Related U.S. Application Data
(60) Provisional application No. 63/088,514, filed on Oct. 7, 2020.

(51) Int. Cl. A61B 18/14; A61B 18/12

(52) U.S. Cl. CPC A61B 18/1492; A61B 2018/00601; A61B 18/1442; A61B 18/1206

(57) ABSTRACT

An elongated medical assembly includes a first elongated medical device configured to be inserted, at least in part, into the patient, and also configured to be maneuvered proximate to the biological feature. A second elongated medical device is configured to be received, at least in part, in the first elongated medical device, and also configured to be selectively extended, at least in part, from the first elongated medical device toward, and proximate to, the biological feature. A biological-removal device is mounted to the second elongated medical device, and configured to cut a portion of the biological feature after the second elongated medical device has been positioned proximate to the biological feature.

### TECHNICAL FIELD

This document relates to the technical field of (and is not limited to) an elongated medical assembly configured to cut a biological feature of a patient (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with existing (known) systems configured to cut a biological feature of a patient. After much study of, and experimentation with, the existing (known) systems configured to cut a biological feature of a patient, an understanding (at least in part) of the problem and its solution have been identified (at least in part) and are articulated (at least in part) as follows:

The transcatheter aortic valve replacement (TAVR) procedure is a minimally-invasive medical procedure to replace a narrowed aortic valve (of the heart) that fails to open properly (aortic valve stenosis). During the TAVR procedure, doctors may insert a catheter into the leg or the chest and guide the catheter to the heart. To address a possible occurrence of Latrogenic Coronary Obstruction (LCO) during the TAVR procedure, the Bioprosthetic Aortic Scallop Intentional Laceration (BASILICA) procedure may be used (for prevention of LCO). The BASILICA procedure involves the use of an electrified guidewire to traverse and then lacerate the aortic leaflet (of the heart). Once the aortic leaflet is lacerated (as a result of executing the BASILICA procedure), the leaflets may then splay away from the coronary ostia and blood flow may be maintained (during the TAVR procedure).

The left ventricular outflow tract (LVOT), which connects to the aorta (of the heart), is nearly indistinguishable from the rest of the ventricle (of the heart, in which the outflow tract is derived from the secondary heart field, during cardiogenesis). The mitral valve replacement (TMVR) procedure involves replacing the diseased mitral valve of a patient's heart with either a mechanical valve or a tissue (bioprosthetic) valve. The mitral valve may need to be replaced because (A) the valve is leaky (mitral valve regurgitation), and/or (B) the valve is narrowed and doesn't open properly (mitral valve stenosis). To address any possible occurrence of obstruction of the LVOT during the TMVR procedure, the Laceration of the Anterior Mitral Valve Leaflet to Prevent LVOT Obstruction (LAMPOON) procedure may be used. The LAMPOON procedure involves the use of an electrified guidewire to traverse and then lacerate the mitral leaflet (of the heart). Once the mitral leaflet is lacerated (as a result of the LAMPOON procedure), the leaflets may then splay away from the LVOT and blood flow may be maintained (during the TMVR procedure).

What may be needed is an improved arrangement (apparatus and/or method) for management of (such as removing, obliterating, etc.) a portion of a biological feature (of a patient, such as a leaflet, a thin piece of tissue, a flap, a polyp located in the intestine, etc. that is positioned inside the body of the patient).

To mitigate, at least in part, at least one problem associated with the existing technology, there is provided (in accordance with a major aspect) an apparatus. The apparatus is for use with a patient having a biological feature. The apparatus includes and is not limited to (comprises) an elongated medical assembly comprising a first elongated medical device configured to be inserted, at least in part, into the patient. The first elongated medical device is also configured to be maneuvered proximate to the biological feature. A second elongated medical device is configured to be received, at least in part, in the first elongated medical device, and also configured to be selectively extended, at least in part, from the first elongated medical device toward, and proximate to, the biological feature. A biological-removal device is mounted to the second elongated medical device, and configured to cut a portion of the biological feature after the second elongated medical device has been positioned proximate to the biological feature.

To mitigate, at least in part, at least one problem associated with the existing technology, there is provided (in accordance with a major aspect) a method. The method is for cutting a biological feature of a patient. The method includes and is not limited to (comprises) inserting, at least in part, a first elongated medical device of an elongated medical assembly into the patient. The method also includes receiving, at least in part, a second elongated medical device of the elongated medical assembly in the first elongated medical device. The method also includes maneuvering, at least in part, the first elongated medical device, with the second elongated medical device received in the first elongated medical device, proximate to the biological feature. The method also includes selectively extending, at least in part, the second elongated medical device from the first elongated medical device toward, and proximate to, the biological feature. The method also includes using a biological-removal device, mounted to the second elongated medical device, to cut a portion of the biological feature after the second elongated medical device has been positioned proximate to the biological feature.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 to FIG. 7B depict side views of embodiments (implementations) of an elongated medical assembly.

The drawings are not necessarily to scale and may be illustrated by phantom lines, diagrammatic representations, and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

| | |
|---|---|
| Medical assembly100 | second catheter 220 |
| first elongated medical device 110 | first catheter 210 |
| first distal section 113 | energy generator 800 |
| second elongated medical device 120 | cable 802 |
| second distal section 123 | patient 900 |
| biological-removal device 130 | biological feature 902 |

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary, or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

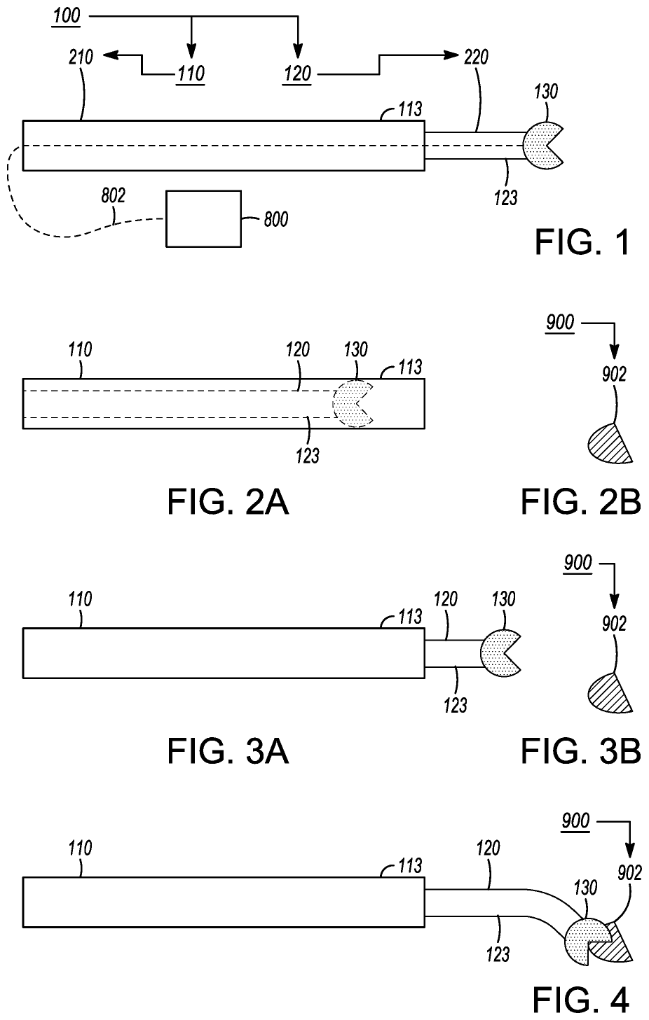

FIG. 1 to FIG. 7B depict side views of embodiments (implementations) of an elongated medical assembly 100.

Referring to the embodiment (implementation) as depicted in FIG. 1, the elongated medical assembly 100 includes a first elongated medical device 110 configured to be inserted, at least in part, into the patient 900, and also configured to be maneuvered proximate to the biological feature 902. The elongated medical assembly 100 also includes a second elongated medical device 120 configured to be received, at least in part, in the first elongated medical device 110, and configured to be selectively extended, at least in part, from the first elongated medical device 110 toward, and proximate to, the biological feature 902. The elongated medical assembly 100 also includes a biological-removal device 130 mounted to the second elongated medical device 120, and configured to cut a portion of the biological feature 902 after the second elongated medical device 120 has been positioned proximate to the biological feature 902.

Referring to the embodiment (implementation) as depicted in FIG. 1, the elongated medical assembly 100 is configured to be inserted into a confined space defined by a living body (the patient 900).

Referring to the embodiment (implementation) as depicted in FIG. 1, the components of the elongated medical assembly 100 includes biocompatible material properties suitable for specific performance (such as, dielectric strength, thermal, electrical insulation, corrosion, water resistance, heat resistance, etc.), for compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment (implementation) as depicted in FIG. 1, some of the components of the elongated medical assembly 100 may include a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus being applied to the shape-memory material. This is known as the shape memory effect (SME). Superelasticity (in alloys) may be observed once the shape-memory material is deformed under the presence (an application) of a stimulus force.

Referring to the embodiment (implementation) as depicted in FIG. 1, the elongated medical assembly 100 is configured for use on a patient 900 having a biological feature 902. The elongated medical assembly 100 includes a first elongated medical device 110 having a first distal section 113 configured to be inserted, at least in part, into the patient 900. The first distal section 113 (of the first elongated medical device 110) is configured to be maneuvered proximate to the biological feature 902; this is done, preferably, after (once) the first elongated medical device 110 has been inserted, at least in part, into the patient 900. The elongated medical assembly 100 also includes a second elongated medical device 120 having a second distal section 123 configured to be received, at least in part, in the first elongated medical device 110. The second distal section 123 (of the second elongated medical device 120) is configured to be selectively extended, at least in part, from the first elongated medical device 110 toward, and proximate to, the biological feature 902; this is done, preferably, after (once) the first distal section 113 of the first elongated medical device 110, in use, has been maneuvered proximate to the biological feature 902. The elongated medical assembly 100 also includes a biological-removal device 130 mounted to the second distal section 123. The biological-removal device 130 is configured to cut a portion of the biological feature 902; this is done, preferably, after (once) the second distal section 123 of the second elongated medical device 120 has been positioned proximate to the biological feature 902.

Referring to the embodiment (implementation) as depicted in FIG. 1, the elongated medical assembly 100 may be used to cut (cut off, remove) at least one portion of the biological feature 902, such as a leaflet (of the heart), a thin piece of tissue, a biological flap, a polyp (positioned in the intestine), etc.

Referring to the embodiment (implementation) as depicted in FIG. 1, the first elongated medical device 110 may include, for instance, a first catheter 210 or any equivalent thereof

Referring to the embodiment (implementation) as depicted in FIG. 1, the second elongated medical device 120 includes a second catheter 220. Referring to the embodiment (implementation) as depicted in FIG. 1, the first elongated medical device 110 includes the first catheter 210. The second elongated medical device 120 includes the second catheter 220.

Referring to the embodiment (implementation) as depicted in FIG. 1, the first elongated medical device 110 (or the first catheter 210) has a usable length that is long enough to reach from the femoral artery to the leaflet (of the heart). The first elongated medical device 110 may have an inner diameter that is able to accommodate the second elongated medical device 120 (of the second catheter 220). The first elongated medical device 110 may be made of polyether block amides or other plastics and/or polymeric materials, silastic materials, silicon-based polymers, polyimides, polyurethanes, polyamides, polyvinylchlorides, polyesters, polyethylenes, polyether ether ketone, perfluoroalkoxy, fluorinated ethylene, propylene, etc. The first elongated medical device 110 may have braiding to improve the transmission of force and torque. The first elongated medical device 110 may be coated with polytetrafluoroethylene (PTFE) to increase lubricity. The first elongated medical device 110 may be radiopaque and/or echogenic to improve medical visualization (via a medical imaging system). The first elongated medical device 110 is configured to be detected by a medical imaging system.

Referring to the embodiment (implementation) as depicted in FIG. 1, the second elongated medical device 120 (or the second catheter 220) may have a usable length that is long enough to reach from the femoral artery to the appropriate leaflet of the heart. The second elongated medical device 120 may be able to accommodate a guidewire (such as, with an inner diameter of about 0.032 inches, etc.). The second elongated medical device 120 may have an outer diameter that fits inside the first elongated medical device 110. The second elongated medical device 120 may be made of polyether block amides, silastic materials, silicon-based polymers, polyimides, polyurethanes, polyamides, polyvinylchlorides, polyesters, polyethylenes, polyether ether ketone, perfluoroalkoxy, fluorinated ethylene, propylene, etc. The second elongated medical device 120 may have braiding to improve the transmission of force and torque. The second elongated medical device 120 may be coated with polytetrafluoroethylene (PTFE) to increase lubricity. The second elongated medical device 120 may have a handle (known and not depicted) that has at least one control device configured to steer the distal tip of the second elongated medical device 120. The second elongated medical device 120 may have another control for moving or maneuvering the jaws of the biological-removal device 130 (such as to close or open the jaws). The biological-removal device 130 includes jaws configured to selectively grab a portion of the biological feature 902. The second elongated medical device 120 may have a pull-ring and pull-wires that run the length of the catheter to facilitate deflection (bending) of the second elongated medical device 120. The deflection mechanism may be bidirectional, with either symmetrical or asymmetrical curves, or unidirectional. The second elongated medical device 120 may have at least one wire that runs a length of the second elongated medical device 120 (the catheter) to provide an electrical connection between an energy emitter (also called an electrode) of the biological-removal device 130 and an electrical connector cable (known and not depicted). The electrode is configured to emit energy (such as radiofrequency energy). The second elongated medical device 120 may be radiopaque and/or echogenic to improve visualization via a medical imaging system. The second elongated medical device 120 is configured to be detected by a medical imaging system.

Referring to the embodiment (implementation) as depicted in FIG. 1, the biological-removal device 130 is configured to hold a portion of the biological feature 902. The biological-removal device 130 is configured to selectively emit energy (such as, radiofrequency energy) toward the biological feature 902 in such a way that the biological-removal device 130, once activated, obliterates (in use) a portion of the biological feature 902 that is held with no remnant pieces of biological tissue free to move within the bloodstream of the patient 900. The biological-removal device 130 may be called a cutting mechanism, etc. The biological-removal device 130 incudes jaws configured to be manipulated (selectively opened and closed). The jaws are configured to selectively grab a portion of the biological feature 902. The biological-removal device 130 is configured to selectively emit energy (preferably, toward the biological feature 902); this is done in such a way that a portion of the biological feature 902 (that is held by the jaws) is obliterated (preferably, with no remnant pieces of biological tissue free to move within the bloodstream of the patient 900). The biological-removal device 130 is configured to electrically connect to an energy generator 800 (via a cable). The energy generator 800 is configured to be electrically connected to the biological-removal device 130 via a cable 802 (via a proximal section of the second elongated medical device 120). The energy generator 800 is configured to allow the physician to control when the electrodes deliver energy (such as radiofrequency energy). The biological-removal device 130 may be shaped and sized depending on the shape of the portion of the biological feature 902 to be removed; for example, the aortic leaflet of the heart may require a different shape and size than the mitral leaflet of the heart, etc. The biological-removal device 130 may be radiopaque and/or echogenic to improve the visualization of the biological-removal device 130 via a medical imaging technique (known and not further described) to make a determination of the correct position (or location) of the biological-removal device 130. The medical imaging system may include, for instance, an electroanatomical mapping system, a fluoroscopy mapping system, etc. The biological-removal device 130 may include an electrode, a sharp piece, an electrified wire, etc., and any combination and/or equivalent thereof.

Referring to the embodiment (implementation) as depicted in FIG. 1, the biological-removal device 130 may, for instance, include an energy emitter, such as an electrode, configured to be used as a radiofrequency puncture device, such as the type used in the BAYLIS (TRADEMARK) POWERWIRE (REGISTERED TRADEMARK) radio frequency guidewire manufactured by BAYLIS MEDICAL COMPANY (headquartered in Canada). In accordance with another embodiment, the biological-removal device 130 may include (and is not limited to) a mechanical cutting portion.

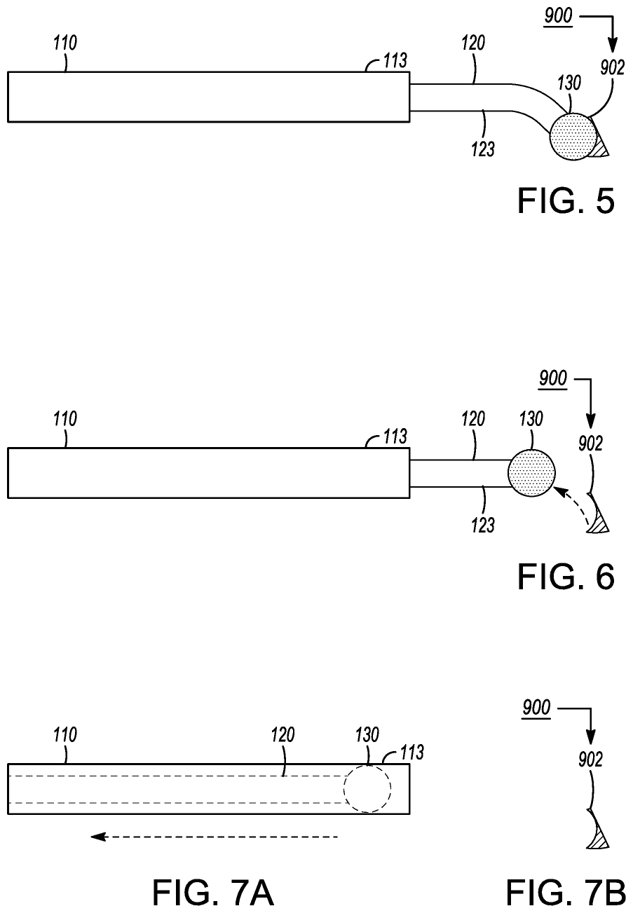

Referring to the embodiments (implementations) as depicted in FIG. 2A to FIG. 7B, there is depicted a method (workflow) of cutting (of how to cut) the biological feature 902 of the patient 900. With reference to FIG. 2A and FIG. 2B, the method includes inserting, at least in part, the first elongated medical device 110 (of the elongated medical assembly 100) into the patient 900. With reference to FIG. 2A and FIG. 2B, the method also includes receiving, at least in part, the second elongated medical device 120 (of the elongated medical assembly 100) in the first elongated medical device 110 (this step might be done before the previous step if desired). With reference to FIG. 3A and FIG. 3B, the method also includes maneuvering, at least in part, the first elongated medical device 110 (with the second elongated medical device 120 received in the first elongated medical device 110) proximate to the biological feature 902. With reference to FIG. 4, the method also includes selectively extending, at least in part, the second elongated medical device 120 from the first elongated medical device 110 toward, and proximate to, the biological feature 902. With reference to FIG. 5, the method also includes using the biological-removal device 130 (in which the biological-removal device 130 is mounted to the second elongated medical device 120) to cut (remove, obliterate, etc.) a portion of the biological feature 902; this is done, preferably, after the second elongated medical device 120 has been positioned proximate to the biological feature 902. With reference to FIG. 6, FIG. 7A and FIG. 7B, the method also includes removing (retracting) the second elongated medical device 120 and the first elongated medical device 110 away from the patient 900.

Referring to the embodiment (implementation) as depicted in FIG. 2A and FIG. 2B, the method includes (preferably) advancing the first elongated medical device 110 (such as, the first catheter 210 as depicted in FIG. 1) into the patient 900 (such as, into the femoral artery of the patient 900).

Referring to the embodiment (implementation) as depicted in FIG. 3A and FIG. 3B, the first elongated medical device 110 continues to be advanced until the first distal section 113 (of the first elongated medical device 110) reaches the biological feature 902 (such as the leaflet, etc.). The first elongated medical device 110 may be retracted (removed) from the biological feature 902; this is done, preferably while the second distal section 123 of the second elongated medical device 120 (having the biological-removal device 130) remains positioned proximate to the biological feature 902). Alternatively, the second distal section 123, of the second elongated medical device 120 (or the second catheter 220 of FIG. 1), having the biological-removal device 130 selectively advanced (moved away) from the first elongated medical device 110 toward a position located proximate to the biological feature 902.

Referring to the embodiment (implementation) as depicted in FIG. 4, the method further includes positioning the biological-removal device 130 (having the jaws or jaw); this is done, preferably, in such a way that the jaws may be positioned, at least in part, around a portion of the biological feature 902 (such as, the leaflet of the heart, etc.). The method further includes positioning the biological-removal device 130 in such a way that the biological-removal device 130 may selectively grab (and hold) a portion of the biological feature 902. The method may further include using a deflection mechanism (known and not depicted) of the second elongated medical device 120 to position the biological-removal device 130 relative to the biological feature 902. The method may further include using a medical imaging technique (known and not further described) to make a determination (identification) of the correct position (or location) of the biological-removal device 130 (before the biological-removal device 130 is used or activated, etc.). The medical imaging system may include, for instance, an electroanatomical mapping system, a fluoroscopy mapping system, etc.

Referring to the embodiment (implementation) as depicted in FIG. 5, the method further include closing the jaws (of the biological-removal device 130) around a portion of the biological feature 902 (such as, the leaflet). The method may further includes using a deflection mechanism (known and not depicted) of the second elongated medical device 120 to deploy (move) the jaws (of the biological-removal device 130). The method may further include selectively emitting energy (such as, emitting an application of radiofrequency energy) from the biological-removal device 130 toward the biological feature 902 (after the jaws have been closed around the portion of the biological feature 902); in this manner, the portion of the biological feature 902 may be entirely obliterated with no remnant (stray) biological pieces free to float within (into) the bloodstream of the patient 900 (thereby avoiding imparting potential harm to the patient 900). The method may further include electrically connecting the cable 802 to the biological-removal device 130 (so that the cable 802 is in electrical communication with the biological-removal device 130), and then selectively activating the energy generator 800 (as depicted in FIG. 1) to urge the emission of energy from the biological-removal device 130.

Referring to the embodiment (implementation) as depicted in FIG. 6, the method may further include selectively retracting the second elongated medical device 120 away from a remaining section of the biological feature 902.

Referring to the embodiment (implementation) as depicted in FIG. 7A and FIG. 7B, the method may further include selectively retracting the second elongated medical device 120 and the first elongated medical device 110 away from a remaining section of the biological feature 902. The method may further include retracting the second elongated medical device 120 with the biological-removal device 130 into the interior of the first elongated medical device 110. The method may further include retracting the first elongated medical device 110 away from patient 900 (with the second elongated medical device 120 retracted into the interior of the first elongated medical device 110 along with the biological-removal device 130). The method may further include continuing with another medical procedure, such as the TAVR procedure or the TMVR procedure.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus for a patient having a biological feature, the apparatus comprising:
an elongated medical assembly, including:
a first elongated medical device having a first distal section configured to be inserted, at least in part, into the patient; and
the first distal section of the first elongated medical device configured to be maneuvered proximate to the biological feature after the first elongated medical device has been inserted, at least in part, into the patient; and
a second elongated medical device having a second distal section configured to be received, at least in part, in the first elongated medical device; and
the second distal section of the second elongated medical device configured to be selectively extended, at least in part, from the first elongated medical device toward, and proximate to, the biological feature after the first distal section of the first elongated medical device, in use, has been maneuvered proximate to the biological feature; and
a biological-removal device mounted to the second distal section; and
the biological-removal device configured to cut a portion of the biological feature after the second distal section of the second elongated medical device has been positioned proximate to the biological feature.

2. The apparatus of claim 1, wherein:
the first elongated medical device includes a first catheter.

3. The apparatus of claim 1, wherein:
the second elongated medical device includes a second catheter.

4. The apparatus of claim 1, wherein:
the first elongated medical device includes a first catheter; and
the second elongated medical device includes a second catheter.

5. The apparatus of claim 1, wherein:
the first elongated medical device is configured to be detected by a medical imaging system.

6. The apparatus of claim 1, wherein:
the second elongated medical device is configured to be detected by a medical imaging system.

7. The apparatus of claim 1, wherein:
the biological-removal device is configured to be electrically connectable to an energy generator.

8. The apparatus of claim 1, wherein:
the biological-removal device is configured to hold the portion of the biological feature.

9. The apparatus of claim 8, wherein:
the biological-removal device is configured to selectively emit energy toward the biological feature.

10. The apparatus of claim 8, wherein:
the biological-removal device is configured to selectively emit energy toward the biological feature in such a way that the biological-removal device, once activated, obliterates, in use, the portion of the biological feature that is held with no remnant pieces of biological tissue free to move within the bloodstream of the patient.

11. The apparatus of claim 10, wherein:
the biological-removal device includes jaws configured to selectively grab the portion of the biological feature.

12. A method of cutting a biological feature of a patient, the method comprising:
inserting, at least in part, a first elongated medical device of an elongated medical assembly into the patient; and
receiving, at least in part, a second elongated medical device of the elongated medical assembly in the first elongated medical device; and
maneuvering, at least in part, the first elongated medical device, with the second elongated medical device received in the first elongated medical device, proximate to the biological feature; and
selectively extending, at least in part, the second elongated medical device from the first elongated medical device toward, and proximate to, the biological feature; and
using a biological-removal device, mounted to the second elongated medical device, to cut a portion of the biological feature after the second elongated medical device has been positioned proximate to the biological feature.

13. A method of cutting a biological feature of a patient, the method comprising:
advancing a first elongated medical device into the patient; and
advancing the first elongated medical device until a first distal section of the first elongated medical device reaches the biological feature; and
positioning a biological-removal device of a second elongated medical device from the first elongated medical device; and
using the biological-removal device to selectively grab a portion of the biological feature.

14. The method of claim 13, further comprising:
selectively emitting energy from the biological-removal device toward the biological feature, which has been grabbed by the biological-removal device.

15. The method of claim 13, further comprising:
selectively retracting the biological-removal device away from a remaining section of the biological feature.

16. The method of claim 13, further comprising:
selectively emitting energy from the biological-removal device toward the biological feature, which has been grabbed by the biological-removal device, so that the portion of the biological feature is entirely obliterated, by the energy that was emitted from the biological-removal device, with no remnant biological pieces free to float within the bloodstream of the patient.
